(19) United States
(12) Patent Application Publication
(10) Pub. No.: US 2025/0375173 A1
(43) Pub. Date: Dec. 11, 2025

(54) METHOD FOR TOUCH-FREE MOTION CONTROL AND MOBILE MEDICAL DEVICE

(71) Applicant: Siemens Healthineers AG, Forchheim (DE)

(72) Inventor: Stephan Kellnberger, Zorneding (DE)

(21) Appl. No.: 19/227,875

(22) Filed: Jun. 4, 2025

(30) Foreign Application Priority Data
Jun. 5, 2024 (DE) 10 2024 205 154.3

(51) Int. Cl. A61B 6/00

(52) U.S. Cl. CPC A61B 6/4405; A61B 6/4441; A61B 6/4476

(57) ABSTRACT

A method is provided for touch-free motion control of a component of a medical device, which component may be moved and/or maneuvered by a motor, using a handle element that is arranged on the device, for example in the form of a handlebar, comprising the following steps: recording measured values by sensors that are arranged on or at the handle element, for example distance and/or position sensors, while the device is in a first operating mode, wherein the first operating mode excludes touch-free motion control, evaluating the measured values with regard to an approach of at least one hand of an operator, switching the device into a second operating mode if the result of the evaluation satisfies a first preset condition, wherein the second operating mode includes touch-free motion control, and wherein the first condition is a hand of the operator coming within a first distance from the handle element from a first approach direction.

### CROSS REFERENCE TO RELATED APPLICATIONS

This application claims the benefit of DE 10 2024 205 154.3 filed on Jun. 5, 2024, which is hereby incorporated by reference in its entirety.

### FIELD

Embodiments relate to touch-free motion control of a component of a medical device, which component may be moved and/or maneuvered by a motor.

### BACKGROUND

Mobile x-ray devices, for example C-arm x-ray devices, that are arranged on device carts and equipped with wheels and may thus be moved over the hospital floor, have a particularly high degree of flexibility in respect of their deployment in different areas or rooms. In operating theaters, mobile x-ray devices are deployed for surgical interventions, for example to the spinal column, in interventional radiology for minimally invasive procedures such as embolization, for example, and in cardiology for minimally invasive procedures to the heart. Further applications include trauma and emergency deployments, orthopedics or pain management, for example.

Mobile x-ray devices must have manual maneuverability which may be easily managed in practice, for example it must be possible for an operator to steer/control and brake the mobile x-ray device. Modern mobile manually controlled x-ray devices have motor-assisted maneuverability in order to facilitate the operation and/or transport of the device by an operator.

In order to maneuver or move such a motorized mobile device during an operative deployment, use is made of handle elements and/or operating elements that have touch-sensitive control elements, for example, and may be provided with a sterile cover so that the hands of the operator may remain sterile. However, operation and control by such handle elements and/or operating elements with a sterile covering is often susceptible to error, the sterile cover is unwieldy and impractical and must be replaced for each deployment.

Every time imaging is required during interventions in the OR, the mobile x-ray device is activated in a sterile environment. Interaction with the imaging system is often necessary and complicated, since the doctor/surgeon/operator is in sterile clothing and cannot touch the touch display. Likewise, whenever the operator has to move or reposition the imaging system, for example if the image plane must be changed or the C-arm must be moved into another position, this must be undertaken by someone other than the doctor/surgeon/operator.

### BRIEF SUMMARY AND DESCRIPTION

The scope of the present disclosure is defined solely by the claims and is not affected to any degree by the statements within this summary. The present embodiments may obviate one or more of the drawbacks or limitations in the related art. Independent of the grammatical term usage, individuals with male, female or other gender identities are included within the term.

Embodiments provide a method that allows a person performing an interventional procedure additionally to maneuver and/or operate the mobile medical device while maintaining sterility in the OR.

The method for touch-free motion control of a component of a medical device, which component may be moved and/or maneuvered by a motor, using a handle element that is arranged on the device, for example in the form of a handlebar, includes the following steps: recording measured values by sensors that are arranged on or at the handle element, for example distance and/or position sensors, while the device is in a first operating mode, the first operating mode excluding touch-free motion control, evaluating the measured values with regard to an approach of at least one hand of an operator and switching the device into a second operating mode if the result of the evaluation satisfies a first preset condition, the second operating mode including touch-free motion control, the first condition being a hand of the operator coming within a first distance from the handle element from a first approach direction.

The method allows an operator (for example a cardiologist, an interventional radiologist or a surgeon) to move a maneuverable medical device easily and safely during an operation or an interventional procedure without touching non-sterile surfaces with their hands, so that the sterility of their hands may be guaranteed during the entire time. By simple hand movements, the operator may “log in” to the operation, i.e. switch the mobile medical device from a first operating mode (not maneuverable/movable) into a second operating mode (maneuverable/movable), without the need to touch the handle element or the handlebar. Once the operator is logged in thus, the mobile medical device may be moved in a touch-free manner, for example by converting hand movements into control signals for a motor drive. The switch from the first operating mode into the second operating mode may be effected by a simple approach from the predetermined first approach direction. This merely requires sensors that are able to register or detect an approach of the hands, i.e. conventional distance or proximity sensors, for example, and possibly also position sensors or image sensors. The method is both intuitive and easy for the operator to perform, may be effected very quickly, and satisfies safety standards. In this way, the medical intervention may be performed in a manner that is quick and kind to the patient, since a complicated resterilization of the hands of the operator is not required between device movements following contact with non-sterile surfaces (handle element).

According to an embodiment, the touch-free motion control is configured in such a way that measured values, for example recorded by the distance or proximity sensors, are evaluated with regard to movement of at least one hand of the operator and converted into control commands for the automatic motion control of the device. The control commands then trigger a device movement that may be effected by a motor. In this way, for example hand movements of the operator may be used for motorized movement of the medical device without any need to touch the operating module.

The mobile medical device has a device cart that may be driven by a motor and, in the second operating mode, the motion control controls a two-dimensional traversing movement of the device cart on a substantially level substrate. It is therefore possible, for example, to activate the traversing movement of the device cart forwards, backwards and sideways (and/or in combination) on the substrate.

According to a further embodiment, the motion control is configured in such a way that a movement of the hand in a direction parallel to the substrate causes a corresponding automatic movement of the device cart. For example, this may be configured such that an approach of the hand towards the handle element in a direction parallel to the substrate causes a receding movement of the device cart in the same direction and/or a receding movement of the hand away from the handle element in a direction parallel to the substrate causes a following movement in the same direction. Hand movements sideways (i.e. along the handle element) parallel to the substrate cause a following sideways movement of the device cart. Combined hand movements cause combined movements of the device cart. A movement of the device cart on the substrate, which movement is easy and intuitive to activate, may be implemented by an operator in this way.

The first approach direction is parallel to the normal of the plane of the substrate. For example, this means that the approach may take place “from above”, i.e. by an approach of the hand towards the handle element from above if the mobile device is intended to maneuver on an level substrate. By virtue of the approach direction having essentially no directional component that is possible for a movement of the device cart, it is possible clearly and unambiguously to distinguish between a movement of a hand for the purpose of logging in and the movement of the hand for a control movement. It is therefore easier for the operator to avoid operating errors or unintentional device movements.

According to a further embodiment, the device is switched back into the first operating mode if a result of a further evaluation of further measured values satisfies at least one second preset condition, that differs from the first preset condition. In this way, it is possible to achieve a simple and reliable logout from the operation of the device, for example when it is intended to terminate the operation. For example, the second condition is a second distance being exceeded in combination with the opposite direction to the first approach direction. Logging out may therefore be effected, for example, by the operator moving their hand upwards away from the handle element. Provision may also be made whereby any instance of a second distance of the hand from the handlebar being exceeded (irrespective in which direction) causes the medical device to be switched into the first operating mode, in order to automatically avoid unintentional operating errors.

According to a further embodiment, the currently active operating mode at any time is indicated by a visual or acoustic indication. It is thereby possible for both the operator and other personnel quickly and reliably to identify the operating mode of the device. Provision may be made for illuminating the display screen in a color that is designated for the operating mode, for example, or a symbol or even text may be displayed, it being alternatively possible also to output a characteristic tone or tone sequence.

According to a further embodiment, provision is additionally made for recording and evaluating measured values that allow gesture control by the hand. The gesture control may activate functions of the device other than movement, i.e. for example display functions, inputs and outputs, or the selection and triggering of measurements.

Embodiments further include a mobile medical device that includes a device cart with motor-assisted maneuverability and that is configured to perform the method described above, having at least one handle element in the form of a handlebar, at least one distance sensor and/or position sensor being arranged on the handlebar or in the immediate vicinity thereof and the sensors being configured to record measured values with regard to an approach of a hand of an operator, an evaluation unit that is configured to evaluate the measured data from the sensors with regard to distance and approach direction of the hand of the operator, and a control unit for activating a switch of the device from a first operating mode into a second operating mode and back as a consequence of the result of the evaluation, and moreover configured to automatically activate a movement of the device cart on a substrate as a consequence of the movement of the hand of the operator.

According to an embodiment, the device takes the form of a mobile C-arm x-ray device. In addition to a device cart, such a device may also have a mobile C-arm that may likewise be activated by the method. According to a further embodiment, the medical device has at least one distance sensor from the following selection: infrared sensor, light sensor, ultrasound sensor, capacitive or inductive sensor, radio-frequency sensor.

### BRIEF DESCRIPTION OF THE FIGURES

FIG. 1 depicts a sequence of the steps of a method for touch-free motion control according to an embodiment.

FIG. 2 depicts a plan view of a handle element with sensors according to an embodiment.

FIG. 3 depicts a side view of a handle element with sensors, a touchpad and approaching hand according to an embodiment.

FIG. 4 depicts a view of a mobile medical device for performing the method according to an embodiment.

### DETAILED DESCRIPTION

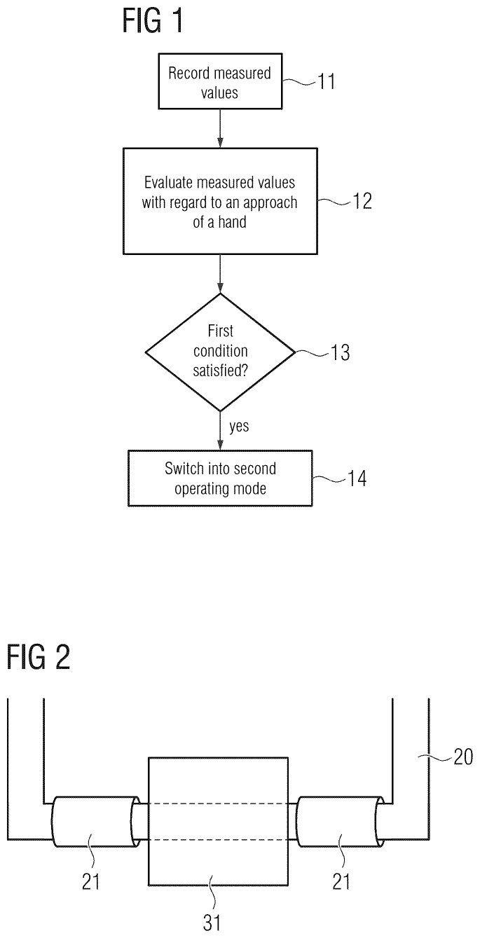

FIG. 1 depicts a sequence of steps of the method for touch-free motion control of a component of a mobile medical device, which component may be moved and/or maneuvered by a motor. Such a component is for example a device cart that may be maneuvered by a motor, is part of the medical device, and allows the medical device to be maneuvered on a substantially level substrate, for example. FIG. 4 depicts a medical device in the form of a mobile C-arm x-ray device 24 that includes a device cart 23 and may be maneuvered by wheels, in the form of Mecanum wheels or Omniwheels 28, that are driven by a motor. Arranged on the C-arm x-ray device 24 or on the device cart 23 is a handlebar 20 for maneuvering/repositioning the device cart 23 using one or more hands manually, semiautomatically or automatically (see also FIG. 2). The C-arm x-ray device 24 includes a C-arm 25 with an x-ray source 26 and an x-ray detector 27 for recording x-ray images of an examination object.

The method may be performed, for example, during a medical intervention or operation using the medical device, i.e. for example when it is undesirable to touch a non-sterile handle element because, for example, the hands of the operator must remain sterile due to the operation.

In a first step 11, measured values are recorded by sensors 21 that are arranged on the handlebar 20 of the medical device. The sensors are, for example, distance or proximity sensors, for example a multiplicity of distance or proximity sensors or a sensor array 32, that are arranged on and/or around the handlebar and which detect or monitor the approach of, for example, one or more hands 22 of an operator. Provision may additionally be made for position sensors or other types of sensors, for example cameras, that ascertain the position of the hand/hands 22. The medical device is initially in a first operating mode, in which the sensors 21 may record the measured values but in which touch-free motion control is also excluded and not supported, or is actively prevented. The recording of measured values by the sensors 21 of the handlebar 20 may be performed continuously and/or “live” during the operation of the device, in order to monitor all hand movements or so that any approaches of a hand may be immediately identified. The measured values are such that they may be used directly or indirectly in a representative manner for determining an approach and/or position of one or more hands.

In a second step 12, the recorded measured values are evaluated with regard to an approach of at least one hand of an operator. This likewise is performed continuously and “live” in order that immediate actions may be taken in response to the evaluation. The respective result of the evaluation may show, for example, whether a hand of an operator is approaching, its current distance, and from which approach direction it is approaching relative to the handlebar 20. The approach direction may be any desired direction, for example. The evaluation may be performed by an evaluation unit, for example, that is part of a system control entity of the medical device.

If the result of the evaluation in the second step 12 satisfies a first preset condition 13, the device is automatically switched into a second operating mode in a fourth step 13. The second operating mode in this case includes the activation of touch-free motion control, that is effected for example by hand movements of the approaching hand.

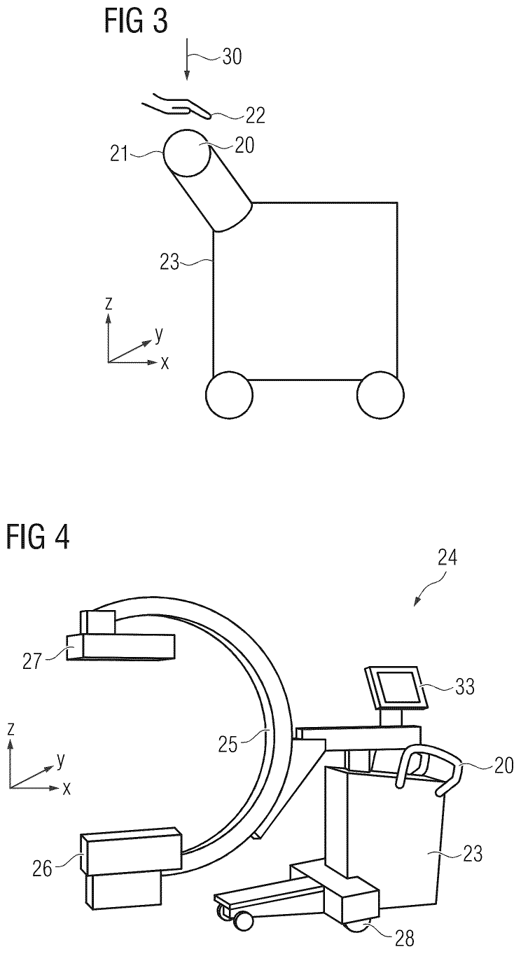

The first preset condition in this case consists essentially of the hand of the operator coming within a first distance from the handlebar (specifically the sensors or the sensor array) from a first approach direction. For example, this may take the form of a hand approaching the handlebar from above (z-coordinate) as shown in FIG. 3, for example if the mobile device is intended to traverse an level substrate. By virtue of the approach direction in this example having no directional component that is possible for a movement of the device cart, it is possible clearly and unambiguously to distinguish between a movement of a hand for the purpose of logging in and the movement of the hand for a control movement. If the hand comes within a specified preset first distance from the handlebar while approaching the handlebar from “above”, the second operating mode is activated, that may also be described as a type of operator “login” into the second operating mode (with the touch-free motion control). As long as the second operating mode is activated, touch-free motion control by the hand is possible. An example of a first distance may be 3 cm or 5 cm or 7 cm, for example.

Provision may further be made whereby a second preset condition must be satisfied in order to return from the second operating mode to the first operating mode. If the hand recedes from the handlebar in the opposite direction to the first approach direction, i.e. for example upwards, and in doing so exceeds a second distance from the handlebar, the medical device is switched back into the first operating mode. Provision may also be made whereby any receding of the hand/hands, irrespective in which direction, causes a switch to the first operating mode, for example as a safety precaution.

In the course of touch-free motion control, hand movements of the operator are used for motorized movement of the medical device without any need to touch the operating module.

In the case of a device cart that may be maneuvered by a motor, the touch-free motion control in the second operating mode allows, for example, a two-dimensional traversing movement of the device cart on a substantially level substrate. It is therefore possible, for example, to activate the traversing movement of the device cart forwards, backwards and sideways (and/or in combination).

The motion control may be configured in such a way that, for example, a movement of the hand in a direction parallel to the substrate (i.e. x-axis or y-axis: forwards, backwards, sideways) activates a corresponding motorized movement of the device cart. For example, this may be configured such that an approach of the hand towards the handle element in a direction parallel to the substrate triggers a receding movement of the device cart in the same direction and/or a receding movement of the hand away from the handle element in a direction parallel to the substrate triggers a following movement in the same direction, i.e. the corresponding control signals for the movement are activated by the system control entity as a consequence of the measurements from the sensors. Hand movements sideways (i.e. along the handle element) parallel to the substrate cause a following sideways movement of the device cart. Combined hand movements cause combined movements of the device cart that, for example in combination with Mecanum wheels of a device cart of a mobile C-arm x-ray device, allow better mobility on the substrate. For example, even a rotation of the device cart could be triggered by moving the hands in opposite directions relative to each other, parallel to the substrate. In such cases, the approach movements or receding movements ascertained by the sensors are registered and converted into control signals for the purpose of activating the motorized movements.

Other components of a mobile medical device, for example a C-arm, may also be activated. In this case, a touch-free motion control described above may be used for spatial repositioning, i.e. for example translation or rotation, of the C-arm. Following activation of the second operating mode as described above, the hand of the operator may cause the component, i.e. the C-arm, likewise to recede or to follow, in this case not only on one plane but also upwards and downwards.

The medical device may show the current operating mode on the display unit 33, for example. For example, provision may be made for coloring the display screen or part of the display screen in a color that is designated for the operating mode, or a symbol or even text may be displayed that symbolizes the respective operating mode. Alternatively, a characteristic tone or tone sequence may be output. The visual or acoustic indication may also be indicated by a simple illuminated element, microphone, or appliance that is carried by the operator and has a communication connection to the device, for example a beeper or an armband. It is also possible to output a haptic signal (for example vibration) to the operator, for example by an armband worn by the operator. The haptic signal may be encoded correspondingly. Such an armband may have a wireless, optical or mechanical communication connection to the device.

Provision may also be made whereby the operator registers with the device by an input before starting the method, so that only the correspondingly registered operator may perform the method. For example, in addition to an input by a user interface, this may also be performed in connection with an armband, special gloves or other appliances having a communication connection to the device. For example, the gloves could therefore be configured in such a way that they are identified by sensors (for example by special optical markers, reflectors, ID chips, etc.). Only the operator having the special gloves may operate the device; other personnel with “regular” gloves cannot log into the device.

In addition to the handlebar 20 and the sensors 21, FIG. 2 also depicts a touch element 31 that is arranged on the handlebar for additional operation by touch. This is however then provided for non-sterile operation.

For the purpose of evaluating the measurement and activating the method, it is possible to use a digital processing unit (DPU), for example, that is implemented in the system control entity of the C-arm x-ray device. The DPU processes the measurements from the sensors and ensures that the corresponding control signals are forwarded to the motors of the device cart or the C-arm.

A further gesture control may also be implemented whereby gestures are recorded, for example by cameras, then evaluated and converted into other control signals for activating the device. An additional voice control may also be implemented.

The sensors may be time-of-flight sensors, for example, that are able to measure the precise absolute distance of the hands from the handlebar. Alternatively, other distance or proximity sensors may also be used, for example infrared sensors, light sensors, ultrasound sensors, capacitive or inductive sensors or radio-frequency sensors may be provided. In addition to this, position sensors may also be used that are able to determine the absolute position of the hands in space, for example cameras.

Independent of the grammatical term usage, individuals with male, female or other gender identities are included within the term.

In order to enable an operator to keep their hands sterile, a method is provided for touch-free motion control of a component of a medical device, which component may be moved and/or maneuvered by a motor, using a handle element that is arranged on the device, for example in the form of a handlebar, including the following steps: recording measured values by sensors that are arranged on or at the handle element, for example distance and/or position sensors, while the device is in a first operating mode, the first operating mode excluding touch-free motion control, evaluating the measured values with regard to an approach of at least one hand of an operator, switching the device into a second operating mode if the result of the evaluation satisfies a first preset condition, the second operating mode including touch-free motion control, and the first condition being a hand of the operator coming within a first distance from the handle element from a first approach direction.

It is to be understood that the elements and features recited in the appended claims may be combined in different ways to produce new claims that likewise fall within the scope of the present disclosure. Thus, whereas the dependent claims appended below depend from only a single independent or dependent claim, it is to be understood that the dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for touch-free motion control of a component of a medical device, wherein the component is moved and/or maneuvered by a motor using a handle element that is arranged on the medical device, the method comprising:
recording measured values by sensors that are arranged on or at the handle element while the medical device is in a first operating mode, wherein the first operating mode excludes the touch-free motion control;
evaluating the measured values with regard to an approach of at least one hand of an operator; and
switching the medical device into a second operating mode when a result of the evaluation satisfies a first preset condition of a hand of the operator coming within a first distance from the handle element from a first approach direction, wherein the second operating mode includes the touch-free motion control.

2. The method of claim 1, wherein the sensors comprise distance or position sensors.

3. The method of claim 1, wherein the touch-free motion control is configured in such a way that measured values recorded by the sensors are evaluated with regard to movement of at least one hand of the operator and converted into control commands for automatic motion control of the medical device.

4. The method of claim 1, wherein the medical device is switched back into the first operating mode when a result of a further evaluation of further measured values satisfies at least one second preset condition that differs from the first preset condition.

5. The method of claim 4, wherein the at least one second preset condition is satisfied when a second distance is exceeded or a second distance is exceeded in combination with an opposite direction to the first approach direction.

6. The method of claim 1, wherein a current operating mode is indicated by a visual or acoustic indication.

7. The method of claim 1, wherein the medical device has a device cart that may be driven by a motor, and the touch-free motion control in the second operating mode activates a two-dimensional traversing movement of the device cart on a substantially level substrate.

8. The method of claim 7, wherein the touch-free motion control is configured in such a way that a movement of the hand in a direction parallel to the substantially level substrate causes a corresponding automatic movement of the device cart.

9. The method of claim 8, wherein an approach of the hand in a direction parallel to the substantially level substrate causes a receding movement of the device cart in a same direction and/or a receding of the hand in a direction parallel to the substantially level substrate causes a following movement in the same direction.

10. The method of claim 1, wherein the first approach direction is parallel to a normal of a plane of a substrate.

11. The method of claim 1, wherein measured values are additionally recorded and evaluated that allow gesture control by the hand.

12. A mobile medical device comprising:
a device cart with motor-assisted maneuverability, the device cart comprising:
at least one handle element comprising a handlebar, wherein at least one sensor is arranged on the handlebar or in an immediate vicinity thereof, wherein the sensor is configured to record measured values with regard to an approach of a hand of an operator;
an evaluation unit that is configured to evaluate the measured values from the sensors with regard to distance and approach direction of the hand of the operator; and
a control unit for activating a switch of the mobile medical device from a first operating mode into a second operating mode and back as a consequence of a result of the evaluation, and further configured to automatically activate a movement of the device cart on a substrate as a consequence of the movement of the hand of the operator.

13. The mobile medical device of claim 12, wherein the mobile medical device is a mobile C-arm x-ray device.

14. The mobile medical device of claim 12, further comprising a movable C-arm.

15. The mobile medical device of claim 12, further comprising:
at least one distance sensor comprising at least one of an infrared sensor, a light sensor, an ultrasound sensor, a capacitive or an inductive sensor, or a radio-frequency sensor.
